(19) United States
(12) Patent Application Publication
(10) Pub. No.: US 2016/0325059 A1
(43) Pub. Date: Nov. 10, 2016

(54) RESPIRATOR STRUCTURE

(71) Applicant: ACARE TECHNOLOGY CO., LTD., New Taipei City (TW)

(72) Inventor: Lin-Ta CHENG, New Taipei City (TW)

(21) Appl. No.: 14/921,846

(22) Filed: Oct. 23, 2015

(30) Foreign Application Priority Data
May 8, 2015 (TW) 104207130

(51) Int. Cl. A61M 16/00; A61M 16/08; A61M 16/16

(52) U.S. Cl. CPC A61M 16/0003; A61M 16/0057; A61M 16/16; A61M 16/0875; A61M 16/0816; A61M 2205/42

(57) ABSTRACT

A respirator structure is provided, which includes a gas supply device. The gas supply device includes a supply device main body, an absorbing vibration module and a gas generating module. The supply device main body is a hollow structure and has an accommodating space and two via holes. The absorbing vibration module is on the accommodating space and has a substrate layer, a flow channel layer, a bearing layer, and a gas generating module. A flow channel layer has a passage and a passage opening. A bearing layer has a bearing part and a bearing opening. The gas generating module has an air inlet and an air outlet. Air flows to the passage through the via holes, the accommodating space and the passage opening, and the gas generating module inhales the air by the air inlet to produce and discharge the air flow outside the supply device main body.

### CROSS-REFERENCE TO RELATED APPLICATION

This application claims the benefit of Taiwan Patent Application No. 104207130, filed on May 8, 2015, in the Taiwan Intellectual Property Office, the disclosure of which is incorporated herein in its entirety by reference.

### BACKGROUND OF THE DISCLOSURE

1. Field of the Disclosure

The present disclosure generally relates to the technical field of the respirator, in particular to a respirator structure having a plurality of vibration absorption structures disposed in a gas supply device.

2. Description of the Related Art

“Snoring” is mainly caused by the upper airway obstruction, which may be obesity, advanced age tissue degeneration, upper airway resistance and so on. When sleeping, such patients may not be able to have a better sleep quality as the muscle of the laryngopharynx becomes soft and hence causes the ptosis of the airway, resulting in that the fresh air cannot be inhaled into the patient's lungs and the oxygen concentration becomes lower gradually. Consequently, the patient may be awakening from time to time as the oxygen deficit of the brain. The syndrome is called “sleep apnea syndrome”. Such syndrome may lead the patient to have a worse sleep quality, and thus, the patient may have headache, drowsiness, attention and memory deficit, anxiety, personality changes, anaphrodisia, etc. in daytime, and snoring, frequent micturition and so on in the evening. Besides, the syndrome may lead to cardiovascular-related diseases easily.

Regarding the treatments of “sleep apnea syndrome”, it can be relieved by means of exercise, weight loss, sleep on one's side, avoid drinking alcohol or taking sleeping pills before going to bed, wearing snore stopper (tooth wear), etc., if the patient has a slight syndrome. However, if the situation is severe, it shall apply surgery or the noninvasive procedures of positive pressure ventilator. The positive pressure ventilator works with mask, achieving the treatment outcome by over 90% successfully. When going to bed, the patient wears the mask working with the positive pressure ventilator, and the pressure produced by the respirator holds the soft respiratory tract to maintain the clearance of the respiratory tract.

The current respirator includes a gas supply device and a humidifying device. The gas supply device produces an air and transports the air to the humidifying device, and then the humidifying device provides the air to the user. However, the internal absorbing vibration structure disposed inside the conventional gas supply device is not good enough because the louder noise produced by the blower worsens the user's sleep quality. In addition, the structure of the conventional humidifying device has the shortcomings which results in that the liquid on the storage device main body flows reversely to the gas supply device. Besides, the devices are not easy to be connected and detached as the complicated structures, resulting in the difficulty of usage.

### SUMMARY OF THE DISCLOSURE

In view of the foregoing technical problems, the objective of the present disclosure provides a respirator structure to resolve the shortcomings of the prior art.

According to one objective of the present disclosure, a respirator structure is provided, which may include a gas supply device. The gas supply device may include a supply device main body, an absorbing vibration module, and gas generating module. The supply device main body may be a hollow shell structure and may have an accommodating space, and a surface of the supply device main body may have a first via hole and one side of the supply device main body may have a second via hole. The absorbing vibration module may be disposed in the accommodating space and the absorbing vibration module corresponding to an internal wall surface of the first via hole of the supply device main body may be disposed with a substrate layer, a flow channel layer, a bearing layer, and a gas generating module sequentially. An internal part near the center of the flow channel layer may have at least one passage, one side of the flow channel layer may have a passage opening, and the passage opening may be connected to the at least one passage and the accommodating space; and a central portion of one surface of the bearing layer may dent towards the other surface of the bearing layer to form a bearing part and then the bearing part may penetrate through the other surface of the bearing layer to form a bearing opening; and one surface of the gas generating module may have an air inlet and one side of the gas generating module may have an air outlet, a part of the gas generating module corresponding to the air inlet may be movably accommodated on the bearing part, and the air inlet may penetrate through and be fixed at the bearing opening correspondingly, and an end of the air outlet may be adjacent to the second via hole. An air may flow to the at least one passage through the first via hole, the accommodating space and the passage opening, the gas generating module may inhale the air in the at least one channel by the air inlet to produce an air flow and may discharge the air flow outside the supply device main body by the second via hole.

Preferably, the bearing layer may include a first bearing substrate adjacent to the flow channel layer and may have the bearing opening. And a second bearing substrate may be disposed on one surface of the first bearing substrate opposite to the flow channel layer, and a center of the second bearing substrate may have the bearing part.

Preferably, an outer periphery of the first bearing substrate may be larger than an outer periphery of the second bearing substrate.

Preferably, a thickness of the first bearing substrate may be larger than a thickness of the second bearing substrate.

Preferably, the bearing substrate may further include a third bearing substrate and a fourth bearing substrate. The third bearing substrate may be disposed between the first bearing substrate and the second bearing substrate and may have a first absorbing opening. The first absorbing opening may communicate the bearing opening and the bearing part. The fourth bearing substrate may be disposed on one surface of the second bearing substrate which is opposite to the third bearing substrate and may have a second absorbing opening. One side of the fourth bearing substrate may have a first output channel and the first output channel may communicate with the second absorbing opening. The bearing part, the first absorbing opening and the second absorbing opening may communicate with each other. When the gas generating module is bore on the bearing part, the first absorbing opening and the second absorbing opening, the air outlet may be disposed in the first output channel.

Preferably, the gas supply device may further include a plurality of positioning modules and an absorbing vibration cover module. The plurality of positioning modules are a rod-shaped structure. One end of each positioning module may be connected to the internal wall surface of the supply device main body and may surround the absorbing vibration module. The absorbing vibration cover module may be a shell structure, a periphery of the absorbing vibration cover module may be detachably connected to the other end of the plurality of positioning modules, one surface of the absorbing vibration cover module may dent inwardly to form a cover part, the cover part may have a vibration reduction layer, and the absorbing vibration cover module may cover a partial periphery of the gas generating module by the vibration reduction layer.

Preferably, the absorbing vibration cover module may further have a vibration absorption layer disposed on one surface of the vibration reduction layer. The vibration absorption layer may have a first cover opening and the other side of the vibration absorption layer may have a second output channel. The second output channel may communicate with the first cover opening. When the vibration reduction layer and the vibration absorption layer of the absorbing vibration cover module cover the partial periphery of the gas generating module, a partial main body of the gas generating module may be disposed in the first cover opening and the air out may be disposed in the second output channel.

Preferably, the gas supply device may further include a gas transport module which is a hollow structure and may be disposed at the second via hole. One end of the gas transport module may be detachably connected to the end of the air outlet, the other end of the gas transport module may be exposed outside the gas supply device main body, and the air flow may flow outside the gas supply device main body through the gas transport module when the gas generating module generates the air flow and discharges the air flow by the air outlet.

Preferably, the respirator structure may further include a humidifying device which includes a storage device main body and a flow guiding module. The storage device main body may be a hollow structure and may have a storage space for accommodating a liquid. One side of the storage device main body may have a first port, a surface of the storage device main body may have a second port, and a predetermined fluid level of the liquid may be higher than the second port. The flow guiding module may be a tube structure and may penetrate and be fixed at the first port. One end of the flow guiding module may have an input port which is disposed outside the storage device main body and may be detachably connected to the other end of the gas transport module, the other end of the flow guiding module may have an output port which is disposed inside the storage space, and the output port may face towards the surface of the storage device main body. When the input port receives the air flow, the air flow may flow to the storage space through the output port to combine with moisture produced by the liquid, and the air flow containing the moisture may flow outside the storage device main body through the second port.

Preferably, one surface and the other surface of the storage device main body may have a predetermined included angle, and the predetermined included angle may be between 20 and 30 degrees.

Preferably, the predetermined included angle may be 25 degrees.

Preferably, the gas supply device may further include a transport device which is a tube structure, and the transport device may have two recesses near both ends outside the transport device and the two recesses may respectively dent towards the center of the transport device gradually. One end of the transport device may be detachably connected to the output port of the gas generating module and the other end of the transport device may be detachably connected to the end of the gas transport module.

Preferably, an internal wall near the both ends of the transport device may respectively have a fastening part formed towards an external wall of the transport device, and the transport device respectively may fasten the ends of the output port and the gas transport module by the fastening part.

Preferably, an extension part may form outwardly along an outer periphery of a side surface at a side of the storage device main body, and the extension part may have a penetration opening corresponding to a partial main body of the surface of the storage device main device, and the respirator structure may further include a buckling device including a press assembly and a connecting assembly. One surface of the press assembly may extrude outwardly to form a press part, the press assembly may be flexibly disposed at the extension part and the press part may penetrate and be fixed on the penetrating opening, and the surface of the press assembly may extend outwardly to form at least one first buckling part adjacent to a side of the press assembly. One end of the connecting assembly may extend outwardly to form at least one connection part, the at least one connecting part may be detachably connected to the other surface of the press assembly and two sides of the other end of the connecting assembly may respectively extend outwardly to form a second buckling part. The side surface of the supply device main body may have at least one first engagement part and a plurality of second engagement parts corresponding to the at least one first buckling part and a plurality of second buckling parts, and the buckling device may detachably buckle the at least one first engagement part and the plurality of second engagement parts by the at least one first buckling part and the plurality of second buckling parts to connect the gas supply device with the humidifying device.

Preferably, the press part may be pressed by an external force to move the at least one first buckling part and the plurality of second buckling parts from a first position to a second position, and when the gas supply device is away from the external force applying to the humidifying device, the gas supply device and the humidifying device may be separated from each other.

As mentioned previously, the respirator structure in accordance with the present disclosure may have one or more advantages as follows.

1. By arranging the absorbing vibration module, the respirator structure is able to absorb the vibration caused by the gas generating module, such that the noise produced in the operation of the gas generating module can be resolve easily.

2. By means of the vibration reduction layer of the absorbing vibration cover module of the respirator structure, the vibration caused in the operation of the gas generating module is absorbed, such that the noise produced in the operation of the gas generating module can be resolved easily.

3. The output port of the flow guide module of the respirator structure is able to be designed to face the surface of the storage device main body to avoid the liquid on the storage device main body flowing reversely to the gas supply device.

4. The transport device of the respirator structure is able to be designed to have two recesses near both ends outside the transport device and the two recesses respectively dent towards the center of the transport device gradually. Hereby, when the gas supply device is inputting the air flow to the humidifying device, the produced noise can be reduced.

5. By means of the buckling device of the respirator structure, the gas supply device and the humidifying device are able to be connected or detached rapidly, so that it becomes easier to use the respirator structure.

### BRIEF DESCRIPTION OF THE DRAWINGS

The detailed structure, operating principle and effects of the present disclosure will now be described in more details hereinafter with reference to the accompanying drawings that show various embodiments of the disclosure as follows.

FIG. 1 is a schematic diagram of the first embodiment of the respirator structure in accordance with the present disclosure.

FIG. 2 is a schematic diagram of the first embodiment of the absorbing vibration module of the respirator structure in accordance with the present disclosure.

FIG. 3 is the first schematic diagram of the first embodiment of the bearing layer of the respirator structure in accordance with the present disclosure.

FIG. 4 is the second schematic diagram of the first embodiment of the bearing layer of the respirator structure in accordance with the present disclosure.

FIG. 5 is a schematic diagram of the second embodiment of the respirator structure in accordance with the present disclosure.

FIG. 6 is a schematic diagram of the third embodiment of the respirator structure in accordance with the present disclosure.

FIG. 7 is a bottom view of the absorbing vibration cover module of the respirator structure in accordance with the present disclosure.

FIG. 8 is a schematic diagram of the fourth embodiment of the respirator structure in accordance with the present disclosure.

FIG. 9 is a schematic diagram of the fifth embodiment of the respirator structure in accordance with the present disclosure.

FIG. 10 is a schematic diagram of the sixth embodiment of the respirator structure in accordance with the present disclosure.

FIG. 11 is a schematic diagram of the clip device of the respirator structure in accordance with the present disclosure.

### DETAILED DESCRIPTION OF THE PREFERRED EMBODIMENTS

Hereinafter, embodiments of the present disclosure will be described in detail with reference to the accompanying drawings so that those skilled in the art to which the present disclosure pertains can realize the present disclosure. As those skilled in the art would realize, the described embodiments may be modified in various different ways, all without departing from the spirit or scope of the present disclosure.

The drawings and description are to be regarded as illustrative in nature and not restrictive. Similar reference numerals designate similar elements throughout the specification.

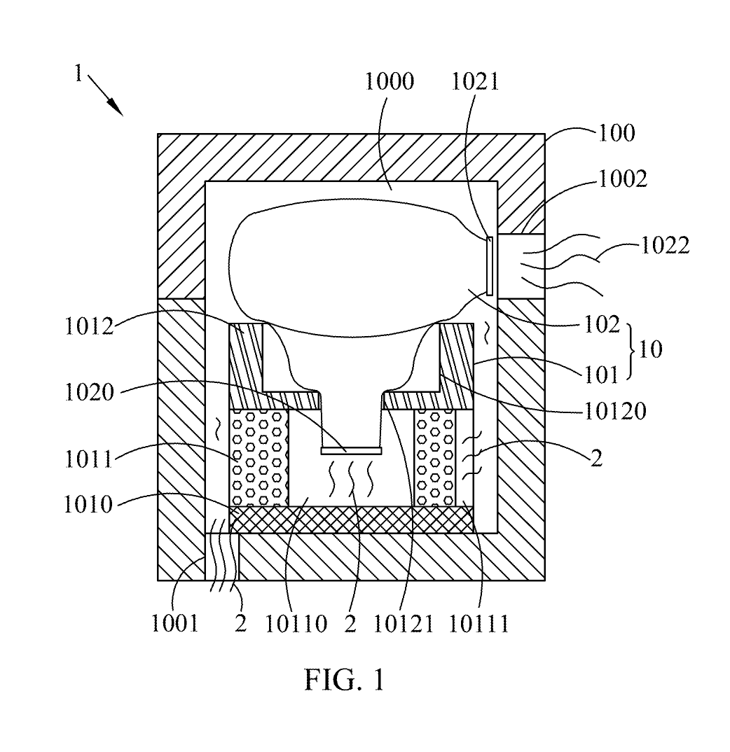

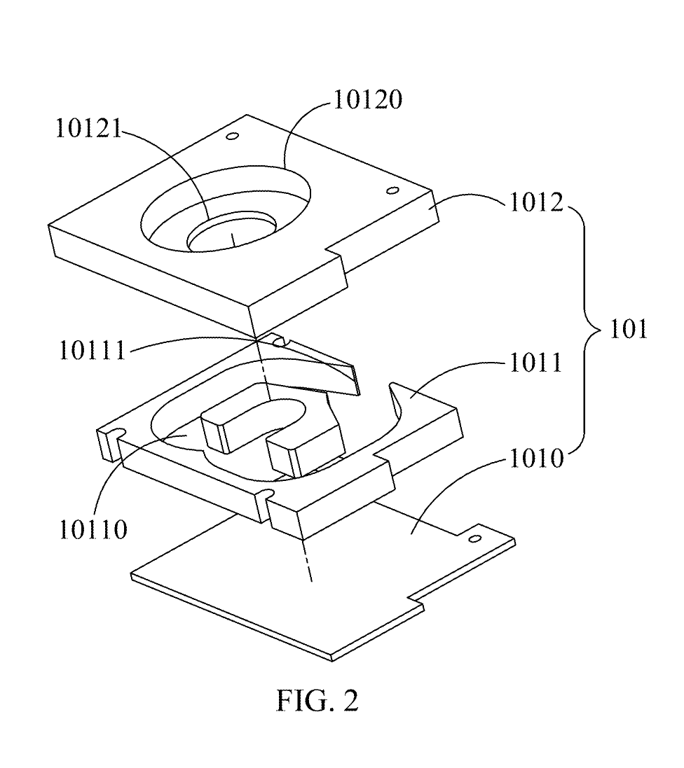

Please refer to FIG. 1 to FIG. 4, which are a schematic diagram of the first embodiment of the respirator structure in accordance with the present disclosure, a schematic diagram of the absorbing vibration module of the respirator structure in accordance with the present disclosure, the first schematic diagram of the bearing layer of the respirator structure in accordance with the present disclosure, and the second schematic diagram of the bearing layer of the respirator structure in accordance with the present disclosure, respectively. As shown in these FIGS., a respirator structure 1 includes a gas supply device 10. The gas supply device 10 includes a supply device main body 100, an absorbing vibration module 101 and a gas generating module 102. The supply device main body 100 is a hollow shell structure and disposed with an accommodating space 1000. One surface of the supply device main body 100 has a first via hole 1001 and the other side of the supply device main body 100 has a second via hole 1002. An absorbing vibration module 101 is disposed in the accommodating space 1000 and the absorbing vibration module 101 corresponding to an internal wall surface of the first via hole 1001 of the supply device main body 100 is disposed with a substrate layer 1010, a flow channel layer 1011, and a bearing layer 1012 sequentially. An internal part near the center of the flow channel layer 1011 has at least one passage 10110, one side of the flow channel layer 1011 has a passage opening 10111, and the passage opening 10111 is connected to at least one passage 10110 and the accommodating space 1000. A central portion of one surface of the bearing layer 1012 dents towards the other surface of the bearing layer 1012 to form a bearing pan 10120 and then penetrates through the other surface of the bearing layer 1012 to form a bearing opening 10121. One surface of the gas generating module 102 has an air inlet 1020 and one side of the gas generating module 102 has an air outlet 1021, a part of the gas generating module 102 corresponding to the air inlet 1020 is movably accommodated on the bearing part 10120, and the air inlet 1020 penetrates through and is fixed at the bearing opening 10121 correspondingly, and an end of the air outlet 1021 is adjacent to the second via hole 1002. An air 2 flows to the at least one passage 10110 through the first via hole 1001, the accommodating space 1000 and the passage opening 10111, and the gas generating module 102 inhales the air 2 in the at least one channel 10110 by the air inlet 1020 to produce an air flow 1022 and discharges the air flow 1022 outside the supply device main body 100 through the second via hole 1002 by the air outlet 1021.

Specifically, the respirator structure 1 of the present disclosure is able to absorb the vibration caused by the gas generating module 102 by means of the absorbing vibration module 101 to resolve the noise caused in the operation of the gas generating module 102, such that it can provide the user with a better sleep environment and improve the user's sleep quality. The gas supply device 10 of the present disclosure includes the supply device main body 100, the absorbing vibration module 101 and the gas generating module 102. The supply device main body 100 is a hollow shell structure and one surface of the supply device main body 100 has the first via hole 1001 connected to the accommodating space 1000 and an outside of the supply device main body 100. One side of the supply device main body 100 has the second via hole 1002 connected to the accommodating space 1000 and an outside of the supply device main body 100. The absorbing vibration module 101 is disposed in the accommodating space 1000, and the absorbing vibration module 101 corresponding to an internal wall surface of the first via hole 1001 of the supply device main body 100 is disposed with the substrate layer 1010, the flow channel layer 1011, and the bearing layer 1012 sequentially. The substrate layer 1010 is at the lowermost layer, the flow channel layer 1011 is in the middle layer and has the at least one passage 10110 near the center, and one side of the flow channel layer 1011 has the passage opening 10111, and the passage opening 10111 is connected to the at least one passage 10110 and the accommodating space 1000. The bearing layer 1012 is at the uppermost layer and a central portion of one surface of the bearing layer 1012 dents towards the other surface of the bearing layer 1012 to form the bearing part 10120 which is able to bear the gas generating module 102, and then the bearing part 10120 penetrates through the other surface of the bearing layer 1012 to form a bearing opening 10121. The bearing opening 10121 and the at least one passage 10110 are communicating with each other. The absorbing vibration module may be the materials characterized of absorbing vibration, such as sponge, silicone rubber, and so on. But it shall be not limited thereto. One surface of the gas generating module 102 has the air inlet 1020 and one side of the gas generating module 102 has the air outlet 1021. A part of the gas generating module 102 corresponding to the air inlet 1020 is movably accommodated on the bearing part 10120, and the air inlet 1020 penetrates through and is fixed at the bearing opening 10121 correspondingly and exposed in the at least one passage 10110. A partial main body of the air outlet 1021 is adjacent to the second via hole 1002, and the gas generating module 120 may be a blower.

When the gas supply device 10 of the respirator structure 1 of the present disclosure is working, an external air 2 enters the accommodating space 1000 by the first via hole 1001 of the supply device main body 100 and flows into the at least one passage 10110 through the passage opening 10111. Then, the gas generating module 102 inhales the air 2 in the at least one passage 10110 by the air inlet 1020 to produce the air flow 1022, and the air outlet 1021 is provided to discharge the air flow 1022 outside the supply device main body 100 through the second via hole 1002. The air outlet 1021 is connected to at least one conduit and a breathing mask, and the air flow 1022 is provided to the user through the conduit and the breathing mask. While the gas generating module 102 is working, the bearing layer 1012 absorbs the vibration caused by the gas generating module 102, and the flow channel layer 1011 is also able to absorb the vibration when the airs are transporting. Hereby, the sounds caused in the operation of the gas generating module 102 is able to be resolved so as to provide the user with a better sleep environment and improve the user's sleep quality.

Furthermore, the bearing layer 1012 of the present disclosure includes a first bearing substrate 1012A and a second bearing substrate 1012B. The first bearing substrate 1012A is adjacent to the flow channel layer 1011 and has the bearing opening 10121. The second bearing substrate 1012B is disposed on one surface of the first bearing substrate 1012A opposite to the flow channel layer 1011, and a bearing part 10120 is disposed in the center of the second bearing substrate 1012B.

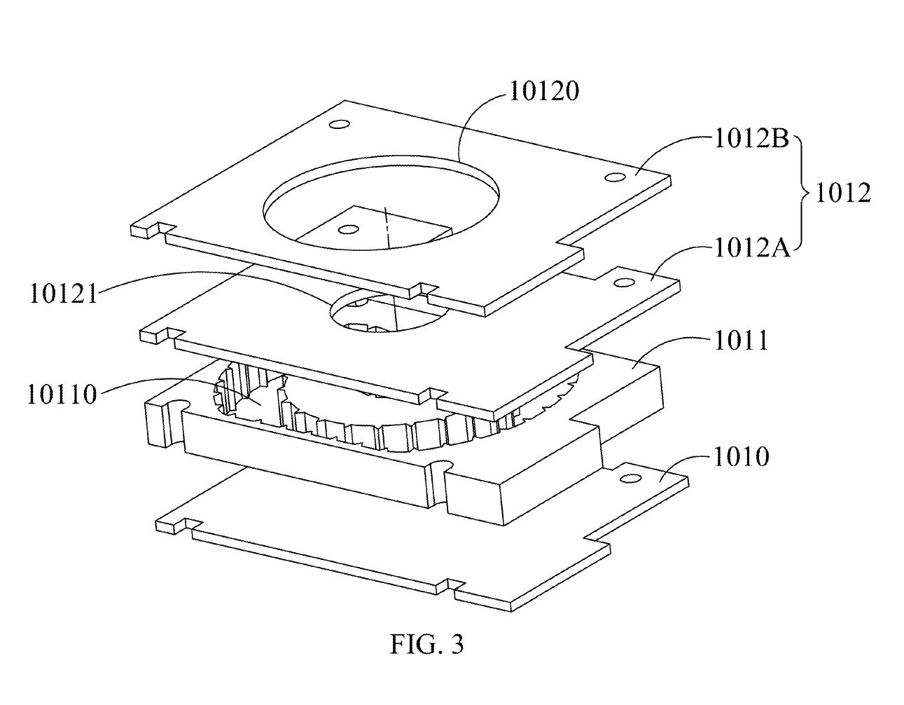

That is to say, the bearing layer 1012 of the present disclosure is further divided into the first bearing substrate 1012A and the second bearing substrate 1012B. As shown in FIG. 3, the first bearing substrate 1012A is on one surface of the flow channel layer 1011 and the bearing opening 10121 is in the center of the first bearing substrate 1012A. The second bearing substrate 1012B is on one surface of the first bearing substrate 1012A opposite to one surface of the channel layer 1011 and the second bearing substrate 1012B is a hollow structure so as to form the bearing part 10120. Hence, when the gas generating module 102 is working, the second bearing substrate 1012B absorbs the vibration caused by the gas generating module 102 while the first bearing substrate 1012A absorbs the vibration when the airs are transporting. Hereby, the vibration caused by the gas generating module 102 can be absorbed more effectively.

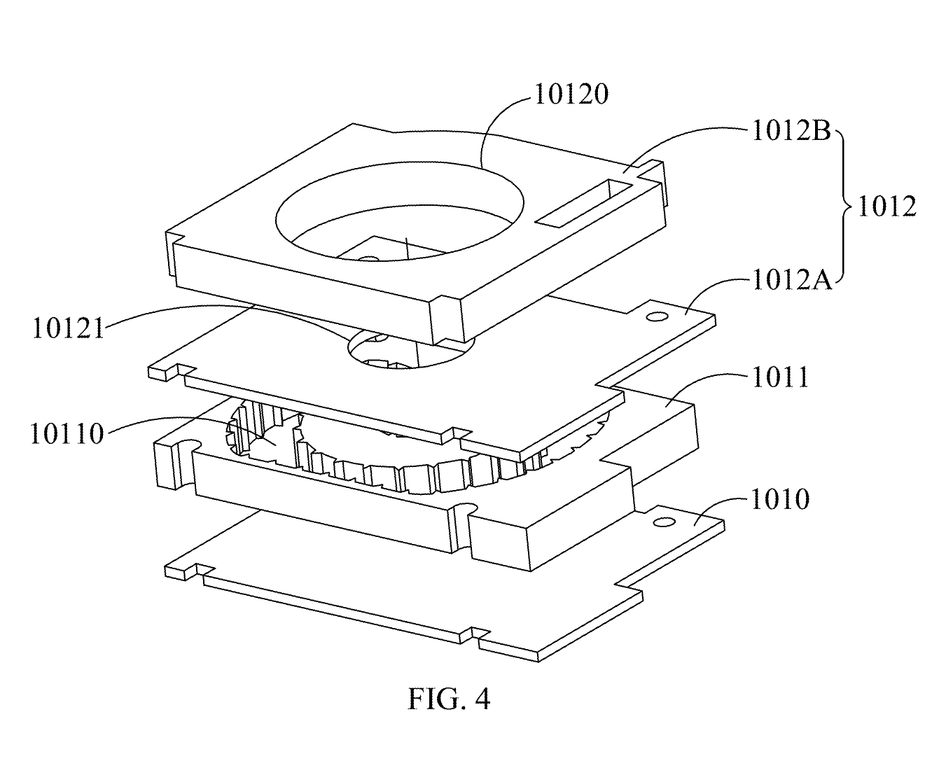

In addition, an outer periphery of the first bearing substrate 1012A of the present disclosure is greater than that of the second bearing substrate 1012B, and a thickness of the first bearing substrate 1012A is smaller than that of the second bearing substrate 1012B. As shown in FIG. 4, the respirator structure 1 of the present disclosure is able to greatly reduce the vibration produced in the operation of the gas generating module 102 by increasing the thickness of the second bearing substrate 1012B so as to mute the operation of the respirator structure 1.

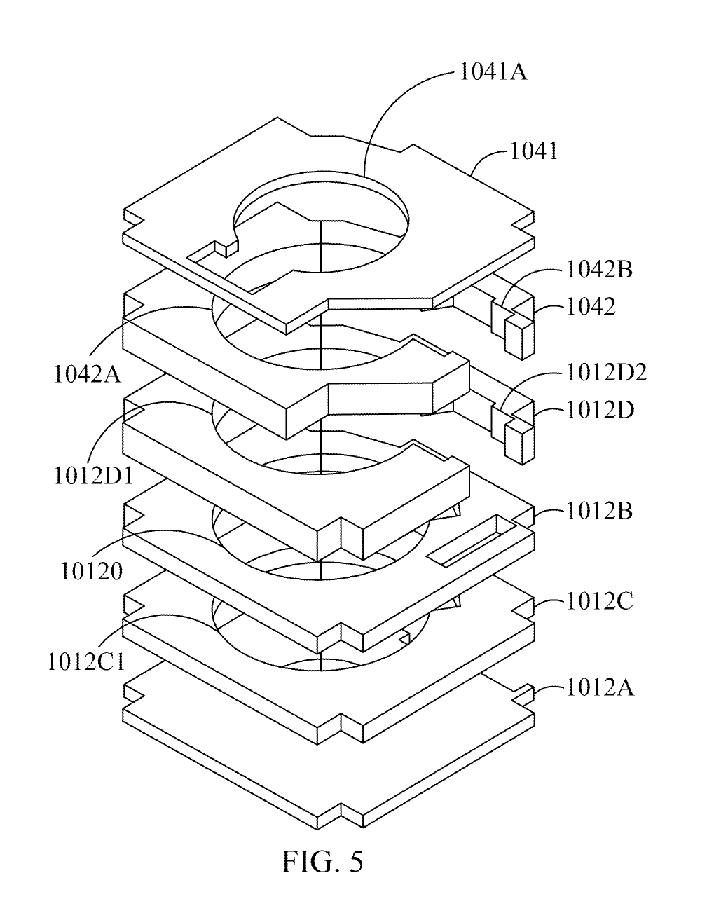

Please refer to FIG. 5 which is a schematic diagram of the second embodiment of the respirator structure in accordance with the present disclosure, and please refer to FIG. 1 to FIG. 4 together. As shown in the figures, the elements used in the respirator structure of the embodiment and that used in the respirator structure of the foregoing embodiments have the similar operation, and the unnecessary details are no longer given herein. However, it is worth mentioning that the bearing substrate 1012 further includes a third bearing substrate 1012C and a fourth bearing substrate 1012D. The third bearing substrate 1012C is disposed between the first bearing substrate 1012A and the second bearing substrate 1012B and has a first absorbing opening 1012C1. The first absorbing opening 1012C1 communicates the bearing opening 10121 and the bearing part 10120. The fourth bearing substrate 1012D is disposed on one surface of the second bearing substrate 1012 B which is opposite to the third bearing substrate 1012C and has a second absorbing opening 1012D1. One side of the fourth bearing substrate 1012D has a first output channel 1012D2 and the first output channel 1012D2 communicates with the second absorbing opening 1012D1. The bearing part 10120, the first absorbing opening 1012C1 and the second absorbing opening 1012D1 communicate with each other. When the gas generating module 102 is bore on the bearing part 10120, the first absorbing opening 1012C1 and the second absorbing opening 1012D1, the air outlet 1021 is disposed in the first output channel 1012D2.

For example, the absorbing vibration cover module 1012 of the present disclosure further includes the third bearing substrate 1012C and the fourth bearing substrate 1012D. The third bearing substrate 1012C is disposed between the first bearing substrate 1012A and the second bearing substrate 1012B and has a first absorbing opening 1012C1. The first absorbing opening 1012C1 communicates the bearing opening 10121 and the bearing part 10120. Besides, the fourth bearing substrate 1012D is disposed on one surface of the second bearing substrate 1012 B which is opposite to the third bearing substrate 1012C and has a second absorbing opening 1012D1. The second absorbing opening 1012D1 communicates with the bearing part 1020. One side of the fourth bearing substrate 1012D has a first output channel 1012D2, and the first output channel 1012D2 communicates with the second absorbing opening 1012D1. Hereby, the bearing layer 1012 designed with a four-layered structure is applied to bear the gas generating module 102 and the first output channel 1012D2 is applied to accommodate the air outlet 1021, such that the noise and the vibration caused by the operation of the gas generating module 102 is reduced so as to provide the user with a better sleep environment and improve the user's sleep quality.

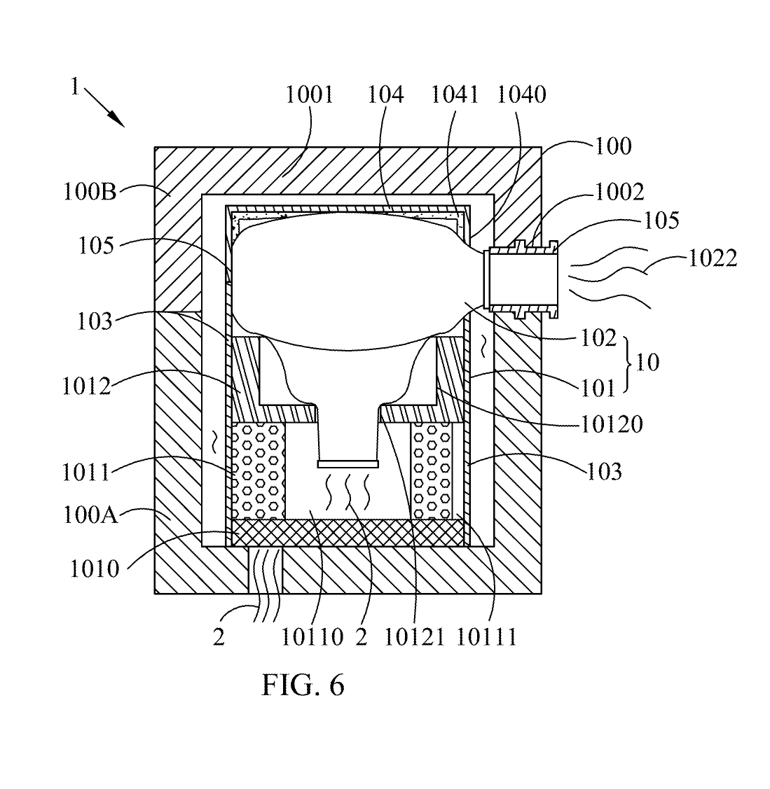

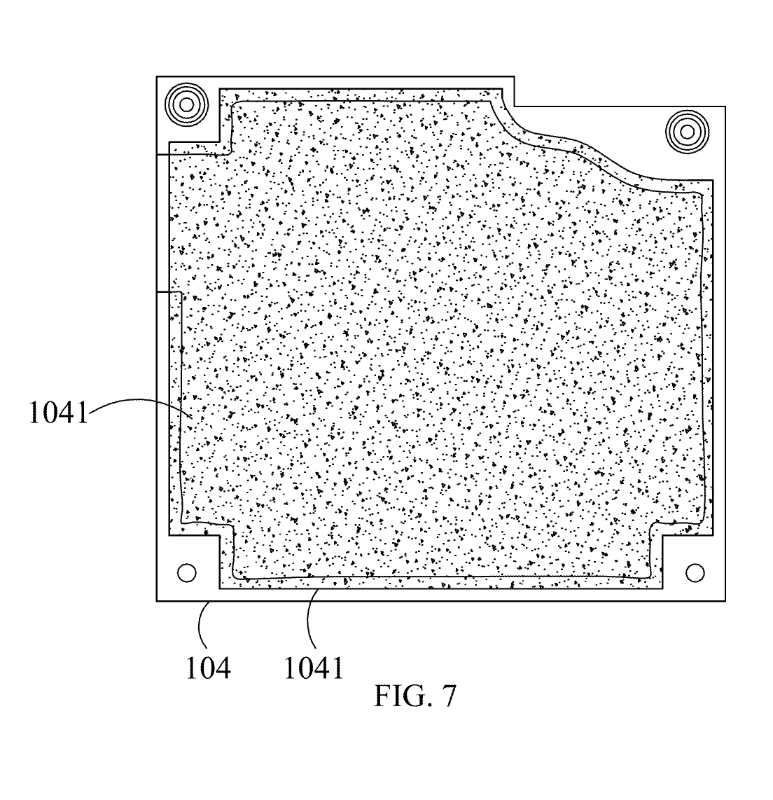

Please refer to FIG. 6 and FIG. 7, which are a schematic diagram of the third embodiment of the respirator structure in accordance with the present disclosure and a bottom view of the absorbing vibration cover module of the respirator structure in accordance with the present disclosure, respectively. Please refer to FIG. 1 to FIG. 5 together. As shown in the FIGS., the respirator structure applied in the embodiment and that applied in the foregoing embodiment have the similar actuation in terms of the members, so the unnecessary details are no longer given herein. However, what has to be addressed is that in the present embodiment, the gas supply device 10 further includes a plurality of positioning modules 103 and an absorbing vibration cover module 104. The plurality of positioning modules 103 are rod-shaped structure and one end of each of the positioning modules 103 is connected to the internal wall surface of the supply device main body 100 and surrounds the absorbing vibration module 101. The absorbing vibration cover module 104 is a shell structure and a periphery of the absorbing vibration cover module 104 is detachably connected to the other end of each of the positioning modules 103. One surface of the absorbing vibration cover module 104 dents inwardly to form a cover part 1040, the cover part 1040 has a vibration reduction layer 1041, and the absorbing vibration cover module 104 covers a partial periphery of the gas generating module 102 by the vibration reduction layer 1041.

For example, the gas supply device 10 of the present disclosure further includes the plurality of positioning modules 103 and the absorbing vibration cover module 104. The plurality of positioning modules 103 are rod-shaped structure and are respectively erected in the accommodating space 1000 of the supply device main body 100 and surround the absorbing vibration module 101. The absorbing vibration cover module 104 is a shell structure which dents inwardly, and a periphery of the absorbing vibration cover module 104 is detachably connected to the other end of each of the positioning modules 103. One surface of the absorbing vibration cover module 104 dents inwardly to form the cover part 1040, the cover part 1040 has the vibration reduction layer 1041, and the absorbing vibration cover module 104 covers a partial periphery of the gas generating module 102 by the vibration reduction layer 1041.

Besides, the absorbing vibration module 101 of the aforementioned embodiment can absorb the vibration caused by the gas generating module 102. When the gas generating module 102 is working, the vibration reduction layer 1041 of the present disclosure is also provided to absorb the vibration caused by the gas generating module 102 to promote the efficiency of the vibration reduction and greatly lower the sounds caused in the operation of the gas generating module 102.

Furthermore, the absorbing vibration cover module 104 further has a vibration absorption layer 1042 disposed on one surface of the vibration reduction layer 1041. The vibration absorption layer 1042 has a first cover opening 1042A and the other side of the vibration absorption layer 1042 has a second output channel 1042B. The second output channel 1042B communicates with the first cover opening 1042A. When the vibration reduction layer 1041 and the vibration absorption layer 1042 of the absorbing vibration cover module 104 are applied to cover the partial periphery of the gas generating module 102, a partial main body of the gas generating module 102 is disposed in the first cover opening 1042A and the air out 1021 is disposed in the second output channel 1042B.

Please refer to FIG. 5 again. The absorbing vibration cover module 104 of the present disclosure further has the vibration absorption layer 1042 disposed on one surface of the vibration reduction layer 1041. The vibration absorption layer 1042 has the first cover opening 1042A and the other side of the vibration absorption layer 1042 has the second output channel 1042B. The second output channel 1042B communicates with the first cover opening 1042A. Hereby, the vibration reduction layer 1041 and the vibration absorption layer 1042 of the absorbing vibration cover module 104 are applied to cover the partial periphery of an upper half of the gas generating module 102 and the second output channel 1042B is applied to accommodate the air outlet 1021, such that the efficiency of reducing the vibration caused by the operation of the gas generating module 102 is promoted. Besides, the vibration reduction layer 1041 also has a second cover opening 1041A and the second cover opening 1041A communicates with the first cover opening 1042A.

It is worth mentioning that the present embodiment is combined with the second embodiment. When the absorbing vibration cover module 104 is connected to the plurality of positioning modules 103, the vibration absorption layer 1042 is stacked on the fourth bearing substrate 1012D and the first cover opening 1042A and the second cover opening 1041A communicate with the second absorbing opening 1012D1. Hence, the vibration reduction layer 1041, the vibration absorption layer 1042 and the bearing layer 1012 designed with a four-layered structure are applied to cover the gas generating module 102, such that the noise and the vibration caused by the operation of the gas generating module 102 can be reduced so as to provide the user with a better sleep environment and improve the user's sleep quality.

Specifically, the gas supply device 10 further includes a gas transport module 105 which is a hollow structure and disposed at the second via hole 1002. One end of the gas transport module 105 is detachably connected to the end of the air outlet 1021, the other end of the gas transport module 105 is exposed outside the gas supply device main body 100, and the air flow 1022 flows outside the gas supply device main body 100 through the gas transport module 105 when the gas generating module 102 generates the air flow 1022 and discharges the air flow 1022 by the air outlet 1021.

That is to say, the gas supply device 10 of the present disclosure further includes the gas transport module 105 which is a disposed at the second via hole 1002 of the gas supply device main body 100. One end of the gas transport module 105 is detachably connected to the end of the air outlet 1021, and the other end of the gas transport module 105 is exposed outside the gas supply device main body 100. As a result, the air flow 1022 flows outside the gas supply device main body 100 through the gas transport module 105 when the gas generating module 102 generates the air flow 1022 and discharges the air flow 1022 by the air outlet 1021. In addition, the other end of the gas transport module 105 is also connected to at least one conduit and a breathing mask, and the user is able to inhale the air flow 1022 transported by the air outlet 1021 through the breathing mask and the conduit.

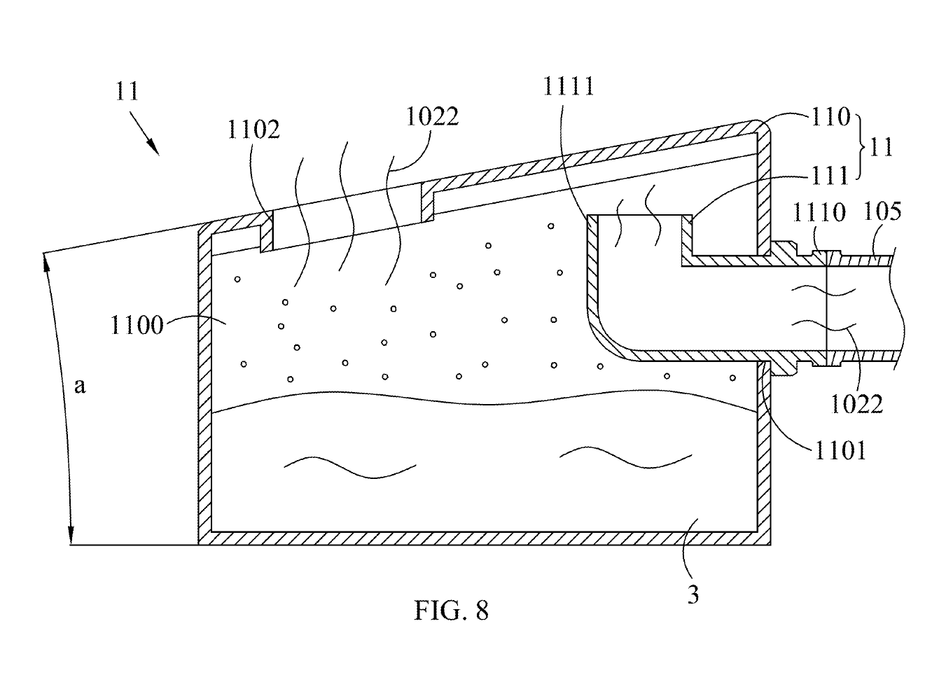

Please refer to FIG. 8, which is a schematic diagram of the fourth embodiment of the respirator structure in accordance with the present disclosure. Please refer to FIG. 1 to FIG. 7 together. As shown in the FIGS., the respirator structure applied in the embodiment and that applied in the foregoing embodiment have the similar actuation in terms of the members, so the unnecessary details are no longer given herein. However, what has to be addressed is that in the present embodiment, the respirator structure 1 further includes a humidifying device 11 which includes a storage device main body 110 and a flow guiding module 111. The storage device main body 110 is a hollow structure and has a storage space 1100 for accommodating a liquid 3. One side of the storage device main body 110 has a first port 1101, a surface of the storage device main body 110 has a second port 1102, and a predetermined fluid level of the liquid 3 is higher than the second port 1102. The flow guiding module 111 is a tube structure and penetrates and is fixed at the first port 1101. One end of the flow guiding module 111 has an input port 1110 which is disposed outside the storage device main body 110 and detachably connected to the other end of the gas transport module 105, the other end of the flow guiding module 105 has an output port 1111 which is disposed inside the storage space 1100, and the output port 1111 faces towards the surface of the storage device main body 110. When the input port 1110 receives the air flow 1022, the air flow 1022 flows to the storage space 1100 through the output port 1111 to combine with moisture produced by the liquid 3, and the air flow 1022 containing the moisture flows outside the storage device main body 110 through the second port 1102.

Specifically, the respirator structure 1 of the present disclosure further includes the humidifying device 11 which includes the storage device main body 110 and the flow guiding module 111. The storage device main body 110 is a hollow structure and has the storage space 1100 for accommodating the liquid 3. The liquid may be water. One side of the storage device main body 110 has a first port 1101 which is communicated with the storage space 1100 and the outside of the storage device main body 110. A surface of the storage device main body 110 further has a second port 1102 which is also communicated with the storage space 1100 and the outside of the storage device main body 110. The liquid 3 in the storage space 1100 has a predetermined fluid level, such as 3 or 5 cm, but it shall be not limited thereto. A predetermined fluid level of the liquid 3 in the storage space 1100 is higher than the second port 1102, and a height of the second port 1102 is lower than that of the first port 1101. Namely, the distance between the predetermined fluid level and the bottom surface of the storage device main body 110 is smaller than that between the second port 1102 and the bottom surface of the storage device main body 110. Similarly, the distance between the second port 1102 and the bottom surface of the storage device main body 110 is smaller than that between the first port 1101 and the bottom surface of the storage device main body 110.

The flow guiding module 111 is a tube structure and penetrates and is fixed at the first port 1101 and extends to the storage space 1100. One end of the flow guiding module 111 has the input port 1110 which is disposed outside the storage device main body 110 and detachably connected to the other end of the gas transport module 105. The other end of the flow guiding module 111 has an output port 1111 which disposed inside the storage space 1100, and the output port 1111 faces towards the surface of the storage device main body 110.

When the gas supply device 10 is using the gas generating module 102 to produce the air flow 1022 and discharges the air flow 1022 by the gas transport module 105, the flow guiding module 111 receives the air flow 1022 by the input port 1110 and transports the air flow 1022 to the storage space 1100 through the output port 1111. Afterwards, the air flow 1022 in the storage space 1100 is combined with the moisture produce by the liquid 3, and the combined gas flows outside of the storage device main body 110 through the second port 1102. The second port 1102 is also communicated with a conduit and a breathing mask, and the user is able to inhale the air flow 1022 containing the moisture which is transported by the breathing mask and the conduit. In addition, if the humidifying device 11 is shook accidentally by the user to lead the liquid 3 in the storage space 1100 to produce vibrations, the liquid 3 fails to flow into the output port 1111 and is unable to flow to the gas supply device 10 as the output port 1111 faces towards the top surface of the storage device main body 110.

Hereby, by means of the arrangement of the flow guiding module 111, the output port 1111 the respirator structure 1 of the present disclosure is designed to face towards the top surface of the storage device main body 110 to avoid the liquid 3 on the storage device main body 110 flowing reversely to the gas supply device 10.

In addition, the humidifying device 11 also includes a heating assembly (not shown in the FIGS.), such as a heating pad. The heating pad is stored at the bottom of the storage device main body 110. When the heating assembly is controlled to heat the bottom of the storage device main body 110 according to signals, the liquid 3 in the storage device main body 110 absorbs the heat energy generated by the heating assembly and produces moisture in the storage space 1100.

In the aforementioned embodiment, the flow guide module 111 is an L-shaped hollow tube structure. Besides, one surface and the other surface of the storage device main body 110 have a predetermined included angle a, and the predetermined included angle a is between 20 and 30 degrees. Preferably, the predetermined included angle a is 25 degrees. As a result, when the moisture produced by the liquid 3 is condensed again on the top surface inside the storage device main body 110, the fluid moves to one end which is away from the output port 1111 along the top surface of the storage device main body 110 to avoid the fluid dropping in the output port 1111.

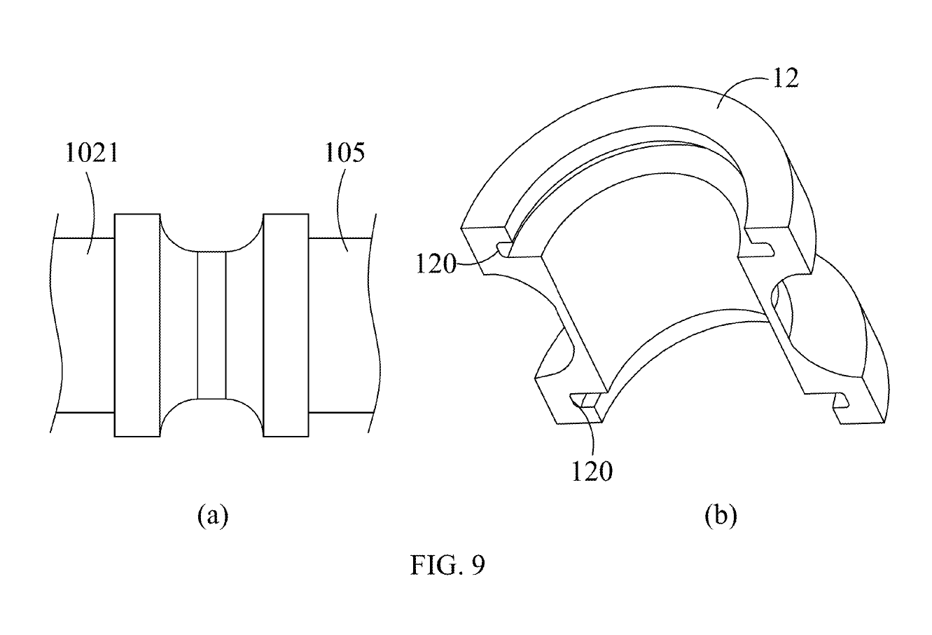

Please refer to FIG. 9 which is a schematic diagram of the fifth embodiment of the respirator structure in accordance with the present disclosure. Please refer to FIG. 1 to FIG. 8 together. As shown in the FIGS., the respirator structure applied in the embodiment and that applied in the foregoing embodiment have the similar actuation in terms of the members, so the unnecessary details are no longer given herein. However, what has to be addressed is that in the present embodiment, the gas supply device 10 includes a transport device 12 which is a tube structure. The transport device 12 has two recesses near both ends outside the transport device 12 and the two recesses respectively dent towards the center of the transport device 12 gradually. One end of the transport device 12 is detachably connected to the output port 1021 of the gas generating module 102 and the other end of the transport device 12 is detachably connected to the end of the gas transport module 105.

For example, the gas supply device 10 of the present disclosure further includes a transport device 12 which is provided to lower the noise when the gas supply device 10 is inputting the air flow 1022 to the humidifying device 11. As shown in FIG. 9 (b), the transport device 12 is a tube structure and has two recesses near both ends outside the transport device 12 and the two recesses respectively dent towards the center of the transport device 12 gradually. Here, the recess is an “I” shape. A thickness between an outer diameter and an inner diameter of two ends is greater than that between an outer diameter and an inner diameter in the middle portion. One end of the transport device 12 is detachably connected to the output port 1021 of the gas generating module 102 and the other end of the transport device 12 is detachably connected to the end of the gas transport module 105.

Hence, as shown in FIG. 9 (a), when the gas generating module 102 transports the air flow 1022 to the humidifying device 11 through the gas transport module 105, the places where the air flow 1022 passes through produce vibrations. By means of the transport device 12, which has a thinner structure in the middle portion, the vibration is absorbed and the noise is also lowered when the air flow 1022 is transporting

Furthermore, an internal wall near the both ends of the transport device 12 of the present disclosure respectively has a fastening part 120 formed towards an external wall of the transport device 12, and the transport device respectively fastens the ends of the output port 1021 and the gas transport module 105 by the fastening part 120. That is to say, the internal wall of both ends of the transport device 12 is disposed with the fastening part 120, respectively to fasten the ends of the output port 1021 and the gas transport module 105 by the fastening part 120, so as to strengthen the communication between the output port 1021 and the gas transport module 105. Ends of the output port 1021 and the gas transport module 105 are disposed with a connecting assembly corresponding to the protrusion of the fastening part 120.

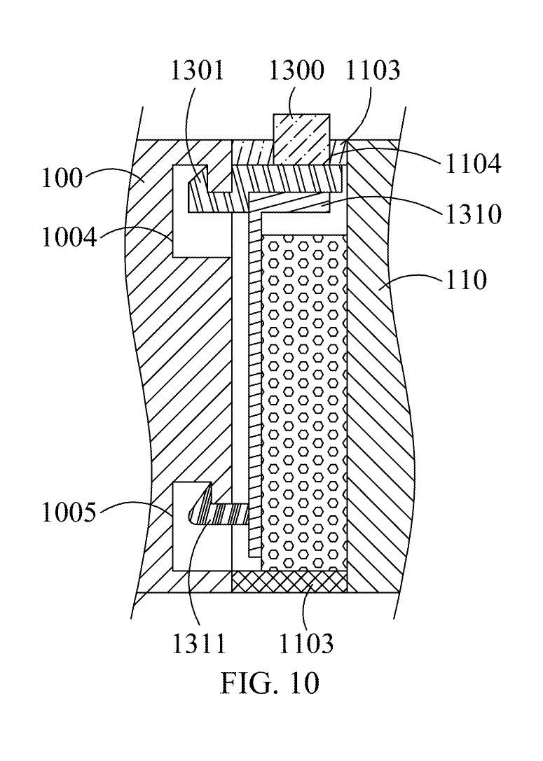

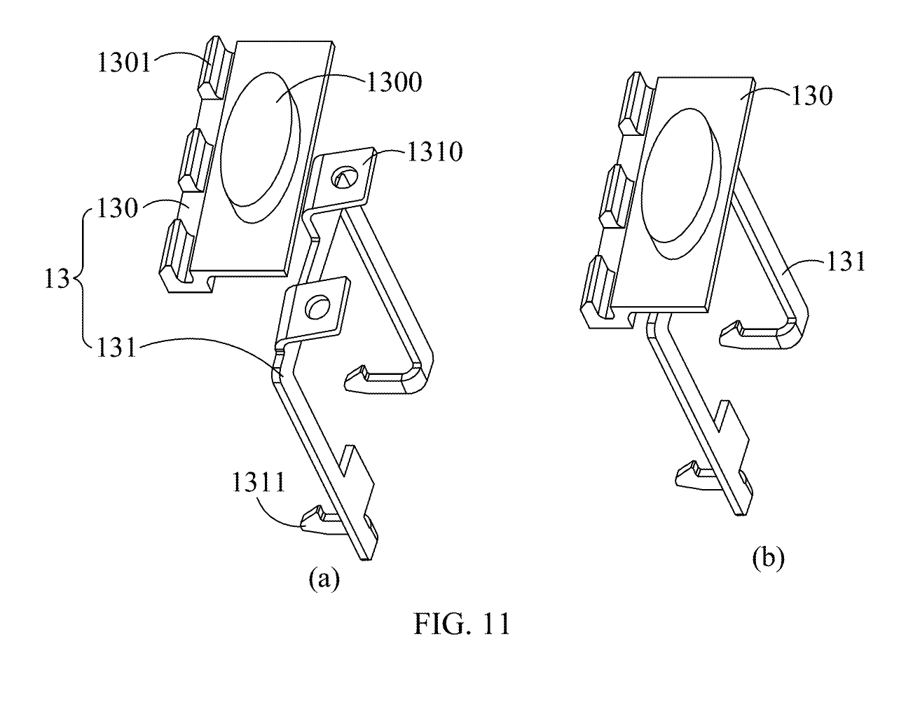

Please refer to FIG. 10 and FIG. 11 which are a schematic diagram of the sixth embodiment of the respirator structure in accordance with the present disclosure and a schematic diagram of the clip device of the respirator structure in accordance with the present disclosure, respectively, and please refer to FIG. 1 to FIG. 9 together. As shown in the FIGS., the respirator structure applied in the embodiment and that applied in the foregoing embodiment have the similar actuation in terms of the members, so the unnecessary details are no longer given herein. However, what has to be addressed is that in the present embodiment, an extension part 1103 forms outwardly along an outer periphery of a side surface at a side of the storage device main body 110, and the extension part 1103 has a penetration opening 1104 corresponding to a partial main body of the surface of the storage device main device 110. The respirator structure 1 further includes a buckling device 13 which includes a press assembly 130 and a connecting assembly 131. One surface of the press assembly 130 extrudes outwardly to form a press part 1300, and the press assembly 130 is flexibly disposed at the extension part 1103. The press part 1300 is flexibly disposed on the penetrating opening 1104, and the surface of the press assembly 130 extends outwardly to form at least one first buckling part 1301 adjacent to a side of the press assembly 130. One end of the connecting assembly 131 extends outwardly to form at least one connection part 1310, the at least one connecting part 1310 is detachably connected to the other surface of the press assembly 130 and two sides of the other end of the connecting assembly 131 respectively extend outwardly to form a second buckling part 1311. Here, the side surface of the supply device main body 100 has at least one first engagement part 1004 and a plurality of second engagement parts 1005 corresponding to the at least one first buckling part 1301 and a plurality of second buckling parts 1311. The buckling device 13 detachably buckles the at least one first engagement part 1004 and the plurality of second engagement parts 1005 by the at least one first buckling part 1301 and the plurality of second buckling parts 1311 to connect the gas supply device 10 with the humidifying device 11.

Specifically, the respirator structure 1 of the present disclosure further includes the buckling device 13 for rapidly connecting or detaching the gas supply device 10 and the humidifying device 11, so as to facilitate the operation of the respirator. As shown in FIGS. 11 (a) and (b), the extension part 1103 forms outwardly along an outer periphery of a side surface at a side of the storage device main body 110, and the extension part 1103 has a penetration opening 1104. One surface of the press assembly 130 of the buckling device 13 extrudes outwardly to form the press part 1300, the press assembly 130 is flexibly disposed at the extension part 1103, and the press part 1300 penetrates and is fixed on the penetrating opening 1104, that is, the press part 1300 is provided to enable the press part 1300 to make the reciprocal displacement in the penetrating opening 1104 on the extension part by a flexible element (not shown in FIGS.), and the surface of the press assembly 130 extends outwardly to form the at least one first buckling part 1301 (a plurality of buckling parts are applied as an exemplary embodiment in the present embodiment) adjacent to a side of the press assembly 130. One end of the connecting assembly 131 of the buckling device 13 extends outwardly to form at least one connection part 1310, the at least one connecting part 1310 is detachably connected to the other surface of the press assembly 130, and two sides of the other end of the connecting assembly 131 respectively extend outwardly to form the second buckling part 1311. Here, the connecting assembly 131 may be an upside-down U shape.

The side surface of the supply device main body 100 is disposed with a plurality of first engagements part 1004 and a plurality of second engagement parts 1005 corresponding to the plurality of first buckling parts 1301 and the plurality of second buckling parts 1311. Hereby, the buckling device 13 detachably buckles the plurality of first engagements part 1004 and the plurality of second engagement parts 1005 by the plurality of first buckling parts 1301 and a plurality of second buckling parts 1311, respectively, so that the gas supply device is connected with the humidifying device 11.

To be more precise, the press part 1300 is pressed by an external force to move the at least one first buckling part 1301 and the plurality of second buckling parts 1311 from a first position to a second position, and when the gas supply device is away from the external force applying to the humidifying device 11, the gas supply device 10 and the humidifying device 11 are separated from each other.

That is to say, if the user wants to detach the gas supply device 10 or the humidifying device 11 to separate the gas supply device 10 from the humidifying device 11, the user applies an external force to the press part 1300 to enable the press part 1300 displacing in the penetrating opening 1104, so as to move the plurality of first buckling parts 1301 (a plurality of buckling parts are applied as an exemplary embodiment in the present embodiment) and the plurality of second buckling parts 1311 from a first position to a second position, that is, the plurality of first buckling parts 1301 and the plurality of second buckling parts 1311 separate from the position of the plurality of first engagements part 1004 and the plurality of second engagement parts 1005. Afterwards, the user separates the gas supply device 10 from the humidifying device 11, and the gas supply device and the humidifying device are separated from each other when the gas supply device 10 is applied the external force.

Contrarily, when the user wants to connect the gas supply device 10 with the humidifying device 11, an external force may be forced to the gas supply device 10 to enable the plurality of first engagements part 1004 and the plurality of second engagement parts 1005 to respectively push the plurality of first buckling parts 1301 and the plurality of second buckling parts 1311, such that the plurality of first buckling parts 1301 and the plurality of second buckling parts 1311 are moved to produce a displacement so as to be engaged with the plurality of first engagements part 1004 and the plurality of second engagement parts 1005, and as a result the gas supply device 10 and the humidifying device 11 are connected with each other.

As mentioned above, the first engagements part 1004 is not limited to one and it may dispose with a plurality of the first engagements parts 1004.

While the means of specific embodiments in present disclosure has been described by reference drawings, numerous modifications and variations could be made thereto by those skilled in the art without departing from the scope and spirit of the disclosure set forth in the claims. The modifications and variations should in a range limited by the specification of the present disclosure.

What is claimed is:

1. A respirator structure, comprising:
a gas supply device comprising:
a supply device main body being a hollow shell structure and having an accommodating space, a surface of the supply device main body having a first via hole and one side of the supply device main body having a second via hole;
an absorbing vibration module disposed in the accommodating space and the absorbing vibration module corresponding to an internal wall surface of the first via hole of the supply device main body disposed with a substrate layer, a flow channel layer, a bearing layer, and a gas generating module sequentially; wherein:
an internal part near the center of the flow channel layer has at least one passage, one side of the flow channel layer has a passage opening, and the passage opening is connected to the at least one passage and the accommodating space; and
a central portion of one surface of the bearing layer dents towards the other surface of the bearing layer to form a bearing part and then the bearing part penetrates through the other surface of the bearing layer to form a bearing opening; and
one surface of the gas generating module has an air inlet and one side of the gas generating module has an air outlet, a part of the gas generating module corresponding to the air inlet is movably accommodated on the bearing part, and the air inlet penetrates through and is fixed at the bearing opening correspondingly, and an end of the air outlet is adjacent to the second via hole;
wherein, an air flows to the at least one passage through the first via hole, the accommodating space and the passage opening, the gas generating module inhales the air in the at least one channel by the air inlet to produce an air flow and discharges the air flow outside the supply device main body by the second via hole.

2. The respirator structure of claim 1, wherein the bearing layer comprises:
a first bearing substrate adjacent to the flow channel layer and having the bearing opening, and
a second bearing substrate disposed on one surface of the first bearing substrate opposite to the flow channel layer, and a center of the second bearing substrate having the bearing part.

3. The respirator structure of claim 2, wherein an outer periphery of the first bearing substrate is larger than an outer periphery of the second bearing substrate.

4. The respirator structure of claim 2, wherein a thickness of the first bearing substrate is larger than a thickness of the second bearing substrate.

5. The respirator structure of claim 2, wherein the bearing substrate further comprising:
a third bearing substrate disposed between the first bearing substrate and the second bearing substrate and having a first absorbing opening, and the first absorbing opening communicating the bearing opening and the bearing part;
a fourth bearing substrate disposed on one surface of the second bearing substrate which is opposite to the third bearing substrate and having a second absorbing opening, one side of the fourth bearing substrate having a first output channel, and the first output channel communicating with the second absorbing opening;
wherein, the bearing part, the first absorbing opening and the second absorbing opening communicate with each other, and when the gas generating module is bore on the bearing part, the first absorbing opening and the second absorbing opening, the air outlet is disposed in the first output channel.

6. The respirator structure of claim 1, wherein the gas supply device further comprises:
a plurality of positioning modules being a rod-shaped structure, one end of each positioning module connected to the internal wall of the surface of the supply device main body and surrounding the absorbing vibration module; and
an absorbing vibration cover module being a shell structure, a periphery of the absorbing vibration cover module detachably connected to the other end of the plurality of positioning modules, one surface of the absorbing vibration cover module denting inwardly to form a cover pan, the cover part having a vibration reduction layer, and the absorbing vibration cover module covering a partial periphery of the gas generating module by the vibration reduction layer.

7. The respirator structure of claim 6 wherein the absorbing vibration cover module further has a vibration absorption layer disposed on one surface of the vibration reduction layer, the vibration absorption layer has a first cover opening and the other side of the vibration absorption layer has a second output channel, the second output channel communicates with the first cover opening, and when the vibration reduction layer and the vibration absorption layer of the absorbing vibration cover module cover the partial periphery of the gas generating module, a partial main body of the gas generating module is disposed in the first cover opening and the air out is disposed in the second output channel.

8. The respirator structure of claim 1, wherein the gas supply device further comprises:
a gas transport module being a hollow structure and disposed at the second via hole, one end of the gas transport module detachably connected to the end of the air outlet, the other end of the gas transport module exposed outside the gas supply device main body, and the air flow flowing outside the gas supply device main body through the gas transport module when the gas generating module generates the air flow and discharges the air flow by the air outlet.

9. The respirator structure of claim 8, further comprising a humidifying device comprising:
a storage device main body being a hollow structure and having a storage space for accommodating a liquid, one side of the storage device main body having a first port, a surface of the storage device main body having a second port, and a predetermined fluid level of the liquid higher than the second port; and
a flow guiding module being a tube structure and penetrating and fixed at the first port, one end of the flow guiding module having an input port which is disposed outside the storage device main body and detachably connected to the other end of the gas transport module, the other end of the flow guiding module having an output port which is disposed inside the storage space, and the output port facing towards the surface of the storage device main body;
wherein, when the input port receives the air flow, the air flow flows to the storage space through the output port to combine with moisture produced by the liquid, and the air flow containing the moisture flows outside the storage device main body through the second port.

10. The respirator structure of claim 9, wherein one surface and the other surface of the storage device main body have a predetermined included angle, and the predetermined included angle is between 20 and 30 degrees.

11. The respirator structure of claim 10, wherein the predetermined included angle is 25 degrees.

12. The respirator structure of claim 8, wherein the gas supply device further comprises a transport device which is a tube structure, and the transport device has two recesses near both ends outside the transport device and the two recesses respectively dent towards the center of the transport device gradually, and one end of the transport device is detachably connected to the output port of the gas generating module and the other end of the transport device is detachably connected to the end of the gas transport module.

13. The respirator structure of claim 12, wherein an internal wall near the both ends of the transport device has a fastening part formed toward an external wall of the transport device respectively, and the transport device respectively fastens the ends of the output port and the gas transport module by the fastening part.

14. The respirator structure of claim 9, wherein an extension part forms outwardly along an outer periphery of a side surface at a side of the storage device main body, and the extension part has a penetration opening corresponding to a partial main body of the surface of the storage device main device, and the respirator structure further comprises a buckling device comprising:
a press assembly, one surface of the press assembly extruding outwardly to form a press part, the press assembly flexibly disposed at the extension part and the press part penetrating and fixed on the penetrating opening, and the surface of the press assembly extending outwardly to form at least one first buckling part adjacent to a side of the press assembly; and
a connecting assembly, one end of the connecting assembly extending outwardly to form at least one connection part, the at least one connecting part detachably connected to the other surface of the press assembly, and two sides of the other end of the connecting assembly respectively extending outwardly to form a second buckling part;
wherein, the side surface of the supply device main body has at least one first engagement part and a plurality of second engagement parts corresponding to the at least one first buckling part and a plurality of second buckling parts, and the buckling device detachably buckles the at least one first engagement part and the plurality of second engagement parts by the at least one first buckling part and the plurality of second buckling parts to connect the gas supply device with the humidifying device.

15. The respirator structure of claim 14, wherein the press part is pressed by an external force to move the at least one first buckling part and the plurality of second buckling parts from a first position to a second position, and when the gas supply device is away from the external force applying to the humidifying device, the gas supply device and the humidifying device are separated from each other.
